(19) United States
(12) Patent Application Publication
(10) Pub. No.: US 2009/0259139 A1
(43) Pub. Date: Oct. 15, 2009

(54) THERMOMASTOGRAPHIC APPARATUS FOR DIFFERENTIATION DIAGNOSTICS FOR DETECTING BREAST PATHOLOGY IN WOMEN AND USE OF THE THERMOMASTOGRAPHIC APPARATUS FOR DIFFERENTIATION DIAGNOSTICS

(76) Inventors: Jacek Stepien, Warsaw (PL); Henryk Jaremek, Warsaw (PL); Grzegorz Pielak, Warsaw (PL)

Correspondence Address: MYERS WOLIN, LLC, 100 HEADQUARTERS PLAZA, North Tower, 6th Floor, MORRISTOWN, NJ, 07960-6834, US

(21) Appl. No.: 12/491,653

(22) Filed: Jun. 25, 2009

Related U.S. Application Data
(63) Continuation of application No. PCT/PL2007/000084, filed on Dec. 21, 2007.

(30) Foreign Application Priority Data
Dec. 29, 2006 (PL) P.381431

(51) Int. Cl. A61B 5/01

(52) U.S. Cl. 600/549

(57) ABSTRACT

A thermomastographic apparatus for differentiation diagnostics for detecting breast pathology in women contains thermosensitive liquid crystals deposited on a flexible, transparent base plate of organic polymer as a foil, comprising simultaneously a carrier and a screen. The apparatus comprises two thermographic matrices, each comprising two test plates for simultaneous examination of both breasts, wherein the first matrix is used for detecting pathophysiological changes of hypothermal expression, and the second matrix is used for detecting pathophysiological changes of hyperthermal expression, in relation to physiological human temperature of 36.6° C., and wherein each test plate is U-shaped and contains at least one monomolecular, continuous layer comprised of a homogenized mixture of liquid crystals exhibiting a thermooptical effect and sealed with elastomer.

This application is a continuation application of International Application No. PCT/PL2007/000084 filed on Dec. 21, 2007, now pending, which claims priority to Polish Patent Application No. P.381431, filed on Dec. 29, 2006, the entire contents of each of which are incorporated herein by reference.

The invention provides a thermomastographic apparatus for differentiation diagnostics for breast pathology detecting in women and use thereof. The invention pertains to the field of medical devices serving for imaging pathological changes in mammary glands of women. In particular, the invention finds its use in early detection breast tumors, including breast cancer.

Use of thermography in breast pathology diagnostics in women was covered by patent applications which employed various different technologies and materials since the early sixties of the 20^{th} century. Thermography as a diagnostic method, along with its physiological basis, is the generally accepted, clinical procedure of imaging pathological changes in mammary glands of women. On Jan. 29, 1982, the US Federal Food and Drug Administration published an official statement along with a classification of thermography, approving thermography as a complementary diagnostic procedure for breast cancer detection. Importance of thermography as a diagnostic method was indicated in: M. Gautherie; Thermobiological assessment of benign and malignant breast diseases. Am. J. Obstet. Gynecol 1983, J. Spitalier, D. Amalric, et al, eds.: The Importance of Infrared Thermography in the Early Suspicion and Detection of Minimal Breast Cancer. Thermal Assessment of Breast Health; MTP Press, 1983, and also in: J. E. Joy, E. E. Penhoet, D. B. Petitti; Saving Women's Lives: Strategies for Improving Breast Cancer Detection and Diagnosis, by Committee on New Approaches to Early Detection and Diagnosis of Breast Cancer, National Cancer Policy Board, Research Council of the National Academies, The National Academies Press, Washington D.C.). N. Eccles in a paper entitled “Thermography—its role in early breast cancer detection and pain monitoring” refers to an abundant scientific output in the field of clinical application of thermography for breast pathology detection, pointing at its high sensitivity and specificity at a level above 90%. In the medical literature index Index Medicus more than 800 separate clinical studies were registered concerning use of thermography in breast pathology diagnostics, more than 300 thousand of women were included into research registered in the database, and some studies involved large populations of from 5,000 (Stark, A., Way, S. The screening of well women for the early detection of breast cancer using clinical examination with thermography and mammography. Cancer 1974), through 16,000 (Haberman, J., Francis, J., Love, T Screening a rural population for breast cancer using thermography and physical examination techniques. Ann NY Acad Sci 1980), 60,000 (Spitalier, H., Giraud, D., et al. Does Infrared Thermography Truly Have a Role in Present-Day Breast Cancer Management? Biomedical Thermology, Alan R. Liss New York, N.Y. 1982) and even 85,000 of female patients (Sciarra, J. Breast Cancer: Strategies for Early Detection. Thermal Assessment of Breast Health; MTP Press, 1983, oraz: Louis, K, Walter, J., Gautherie, M. Long-Term Assessment of Breast Cancer Risk by Thermal Imaging. Biomedical Thermology. Alan R. Liss Inc. 1982), with some of observations included in research lasting up to 12 years. Breast thermography can indicate first symptoms of breast cancer formation preceding by even 10 years any changes indicated by other diagnostic procedures, and when associated with other methods (clinical examination+mammography+thermography) allows to discover 95% of early phases of breast cancer. Clinical research indicates that breast thermography significantly increases a long-term survival rate index in women connected with early detection of breast cancer, even up to 61%. A pathological thermogram may be acknowledged as an autonomous marker of high risk of breast cancer development, and reproducible pathological thermograms connected with 22 times higher risk of breast cancer development in the future. Thermography is recommended as a diagnostic method of choice in regular anticancer monitoring in women with family positive medical history of breast cancer. As I. Nyirjesy indicated in Nyirjesy I., Ayme Y, et al; Clinical Evaluation, mammography and thermography in the diagnosis of breast carcinoma. Thermology 1986, on analysis of retrospective results of the most widely conceived research on thermography, pathological thermograms were noted in minimum 71% to maximum 93% cases of female patients with breast cancer, which signifies very high diagnostic and prognostic credibility.

Physiological foundations for the use of thermography in medical imaging diagnostics comprise a dermothermal effect described in research papers, where a close relationship between expression of thermal anomalies encountered during thermographic examination on a surface of mammary glands, and prevalence of determined types of breast pathology has been demonstrated (Sterns E E, Zee B, SenGupta S, Saunders F W.; Thermography. Its relation to pathologic characteristics, vascularity, proliferation rate, and survival of patients with invasive ductal carcinoma of the breast; Cancer, 1996, a także: Jones C H, Greening W P, Davey J B, McKinna J A, Greeves V J. Thermography of the female breast: a five-year study in relation to the detection and prognosis of cancer. Br J. Radiol. 1975). Similarly, Lawson, Chughtai et al. of McGill University (Lawson R.: Implications of Surface Temperatures in the Diagnosis of Breast Cancer. Can Med Assoc Journ. 75) demonstrated in their pioneering intraoperative research that an increase in superficial temperature of a mammary gland correlated with presence of tumor, is a result of transvascular convection proceeding dynamically within limits of vascular plexuses originating from neoangiogenesis. Vascular proliferation or neoangiogenesis (after: Yahara T. Koga T. Yoshida S, Nakagawa S, Deguchi H, Shirouzu K. Relationship between microvessel density and thermographic hot areas in breast cancer. Surg Today. 2003 także: Gautherie M, Haehnel P, Walter J P, Keith L G.; Thermovascular changes associated with in situ and minimal breast cancers. Results of an ongoing prospective study after four years. J Reprod Med. 1987) is one of the most important pathophysiological markers of a neoplastic process (Guidi A. J., Schnitt S. J.: Angiogenesis in pre-invasive lesions of the breast. The Breast J., 1996) combined with logarithmically growing perfusion demand of abnormal tissue. Neoangiogenesis starts on the very early stage of tumor growth, when its size does not exceed 150 μm (0.15 mm) and it is particularly intense on a stage of a diameter about 1-2 mm. During its dynamical growth, tumor tissue produces significant amounts of nitrous oxide (Rodenberg D A, Chaet M S, Bass R C, Arkovitz M D and Garcia B F. Nitric Oxide: An overview. Am J Surg, 1995. and also: Thomsen L L, Miles D W, Happerfield L, Bobrow L G, Knowles R G and Mancada S. Nitric oxide synthase activity in human breast cancer. Br J Cancer 72, July 1995) having vasodilatory activity, which due to local perfusion exhibits dilating effect on adjacent blood vessels, followed also by a focal temperature increase (Draper J.; Skin Temperature Distribution over Veins and Tumors, Phys Med Biol 16(4), 1971, and: Chao. J.; Measurement of Thermal Properties of Growing Tumors, Poc NY Acad Sci, 1980). Anbar et al. (Anbar M.: Breast Cancer. In: Quantitative Dynamic Telethermometry in Medical Diagnosis and Management. CRC Press, Ann Arbor, 1994) in cross-sectional studies indicated biochemical-immunological cascade of processes involving nitrous oxide synthetases (NOS—nitric oxide synthase), both in a tissue form (constitutive form: c-NOS), localized in endothelial cells, and also in an inducible form (inducible form: i-NOS), the activity of which associated with a size of a neoplastic tumor and its metabolic rate, compounded also with focal tissue temperature growth. A dermothermal effect associated with growth of neoplastic tumors results from numerous overlapping pathophysiological effects, with a dominant role of the above-mentioned angiogenesis process, which found a solid confirmation in in-vivo and in-vitro assays (Love, T.: Thermography as an Indicator of Blood Perfusion. Proc NY Acad Sci Journ. 1980). P. Gamagami (Gamagami P: Indirect signs of breast cancer: Angiogenesis study. In: Atlas of Mammography, Cambridge, Mass., Blackwell Science, 1996) in a widely quoted reference entitled “Atlas of Mammography—novel early symptoms of breast cancer” of 1996, observes that hipervascularization accompanied by hyperthermia, which is seen on thermograms as focal changes of increased temperature, can be noticed in 86% of impalpable breast cancer cases, and remarks that in at least 15% of such cases the thermography may be a method of choice for their detection, especially in cases of tumors which are non-detectable by classical mammography (Sterns E E, Zee B. Thermography as a predictor of prognosis in cancer of the breast, Cancer, 1991). In cases of degenerative processes, such as fibrocystosis, hypothermal loci are visible in thermographic imaging. Goldberg et al. (Goldberg I M, Schick P M, Pilch Y. Shabot M M. Contact plate thermography: a new technique for diagnosis of breast masses. Arch Surg. 1981, also: Sterns E E. The abnormal mammogram in women with clinically normal breasts. Can J. Surg. 1995) indicates that a pathophysiological mechanism for appearance of hypothermal loci in a case of non-neoplastic changes is different from the one presented above and it is connected with an opposite effect—vasoatrophy caused by fibrotic processes and calcification—herein, thermographic changes may exhibit a character of tiny disseminated foci less than 1 mm in diameter and with a temperature lower than the one in adjacent tissue, invisible in classical imaging by the x-ray mammography technique. It is essential for thermographical applications that clinical studies have shown relative indepencence of expression of atypical thermal changes on mammary gland surfaces from cyclic hormonal changes which influence woman's general body temperature, from physiological circadian oscillations of locally measured temperature (Wilson D W, Griffiths K, Halberg F. Simpson H W, Griffiths R, Kemp K W, Nix A B, Rowlands R J. Breast skin temperature rhythms in relation to ovulation. Chronobiologia. 1983, także: Wilson D W, George D, Mansel R E, Simpson H W, Halberg F. Griffiths K. Circadian breast skin temperature rhythms: overt and occult benign and occult primary malignant breast disease. Chronobiol Int. 1984 także: Phillips M J, Wilson D W, Simpson H W, Fahmy D R, Groom G V, Phillips M E, Pierrepoint C G, Blamey R W, Halberg F. Griffiths K Characterisation of breast skin temperature rhythms of women in relation to menstrual status. Acta Endocrinol (Copenh). 1981).

Diagnostic methods are known which make use of remote and contact thermography. In case of the contact thermography, a thermooptical effect resulting from properties of a thermotropic mesophase of liquid crystals is employed, which is caused by a phase transition in a liquid crystal mixture and a change in a fourth order structure due to absorbing a specific amount of electromagnetic radiative (thermal) energy in the infrared range, followed by a resulting change in an optical rotation angle of molecules in individual liquid crystal fractions which form a molecular layer on a surface of a tester plate; the effect revealing itself in a precisely defined temperature, and allowing to calibrate precise a color-temperature response scale.

However, earlier patent applications pertaining to thermodiagnostic devices employing liquid crystals, and a patent to A. Colombo (GB2060879) granted in 1981, and a patent to E. Cassin (EP0059328) granted in 1982 in particular, did not take into account in greater detail a problem of unequivocal readings of thermal changes having certain diagnostic value, by providing indispensable discrimination of colors for particular registered temperatures, which was a source of significant difficulties or even precluded their practical use. Another problem connected with quality of color organ temperature imaging by improperly homogenized liquid crystal mixtures used in devices for thermographic diagnostics, was use of a method of initial sealing of liquid crystals by microencapsulation, consisting in enclosing them in polymer or gelatin microcapsules. On a temperature readout screen, microencapsulated liquid crystals form granules clearly visible with a naked eye, with sizes depending not only on the mere diameter of microcapsules used, but also on a high risk of irregular distribution of particular liquid crystal fractions on a test plate surface (this may also be caused by a lack of earlier dehydration or deionization of a base support plate for liquid crystals). It must be emphasized that, in case of use of microcapsules (as it was disclosed in the patent applications of A. Colombo and E. Cassin), and in particular polypeptide microcapsules, sealing of liquid crystals is additionally compounded by a risk of microcoagulation and forming of macroscopically visible conglomerates, which distort thermographic image with artifacts to the extent excluding, in principle, use in precise (early) medical diagnostics.

D. H. Baltzer, in his invention of 1971 (U.S. Pat. No. 3,620,889) discloses an apparatus employing microencapsulated liquid crystals supported on a forming polymer layer, a complex structure being a proper diagnostic device. Unfortunately, both the above-mentioned technique of liquid crystal microencapsulation employed therein, and unsolved problem of providing uniform dispersion and protecting the liquid crystal layer, precluded wider use of the invention. The greatest obstacle was however inappropriate approach to a solution of the liquid crystal film sealing problem by first forming of a polymer support for liquid crystals, followed by an effort of sealing with a transparent medium, leading inevitably to distortion of the ultrathin and very susceptible to physical damages liquid crystal layer, as well as to non-physiological setting of a color-temperature response scale of liquid crystals; three color transitions with the same color appeared on the test plate screen for distinct temperatures (“blue” for 31° C. and 33.5° C. and 35° C.). In case of employing only one measuring matrix, the situation would lead to artefactual readouts; any unequivocal mapping of a color-temperature liquid crystal response would require use of multiple individual measuring plates separated in non-physiological temperature ranges. The problem of temperature separation of liquid crystal fractions was already analyzed in an earlier invention by J. Fergason and T. Vogl (U.S. Pat. No. 3,114,836), though so far striving to employ already known liquid crystal mixtures prevails over search for new solutions.

In almost all studies on clinical use of thermography, a problem of physiological delimiting of thermographic changes into hypo- and hyper-thermal ones. In case of contact thermography, the problem of separating thermal anomalies is important insomuch as it is not possible to selective eliminate particular thermal phases from a thermogram during examination; therefore, in spite of a very high resolution of the measurement, some difficulties may appear in correctly assessing visually the observed temperature changes and unequivocally identify a pathology, if on one test plate a wide temperature spectrum in a range above and below 36.6° C. is analyzed. Such situation may also be caused by presence of many color transitions (in a blue color spectrum range in particular) described above, which are not unequivocally assigned to one temperature. No satisfactory solution to this significant disadvantage of contact thermography was disclosed until the present application.

In many patents, a problem of bi-uniform correspondence of mapping of mammary gland superficial temperature changes was attempted to be solved by constructing complex multipoint sensory matrices, most frequently circular matrices, containing a great number of thermosensitive elements on a unit of test surface, to register a momentary temperature. Such inventions were e.g.: an invention disclosed in an application filed in 2000 by D. van Hollen (U.S. Pat. No. 6,086,247) and employing electronic microsensors, or an earlier invention disclosing a device similar in shape, but based on detecting temperature changes with liquid crystals, disclosed in an application filed by Z. Sagi (U.S. Pat. No. 4,624,264). Both solutions, apart of their technical limitations connected with tester plate size scaling and low effective resolution, were first of all intended for topographic detection (localization) of thermal anomalies, which seemed aimless in the light of the lack of the mentioned shape optimization, and strictly contact character of the tester, because thermal change indications vanish on tester removal, and precise determination of an observed anomaly location directly on an organ is impossible.

Another problem connected with practical use of contact liquid crystal thermography was the inability to overcome a design constraint of a tester to map in the best possible way temperature distribution on an entire mammary gland surface. A barrier in this case is constituted by the very anatomical structure of a breast, irregularity of its shape and large constitutional diversity in the female population. Many inventions were attempting to break the impasse by designing specific thermographic devices with shapes adapted to anatomical constitution of a breast. A specific case is a patent application of 1977 by E. Flamm (GB 1462413) which discloses a contact thermographic detector in a form of a classical brassiere, employing a liquid crystal layer placed in flexible test plates formed as cups of a common brassiere. A serious technical problem appeared when creating the most important part of the apparatus, namely a liquid crystal detector (to render the liquid crystal layer resistance against mechano-optical distortion and prevent any artifactual readouts.

A similar concept of brassiere underwear with the purpose of contact thermography, this time employing matrices of thermo-electronic sensors, was disclosed in an invention by A. Simpson (GB 2,203,250), and again A. Simpson (GB 1,490,803 and U.S. Pat. No. 4,055,166). Said inventions cannot attain a practical registering resolution sufficient in the case of tumorous changes in situ of a diameter up to 1 mm, since it would entail use of temperature distribution analysis algorithms around sensors based on rules of fuzzy logic; it would require separate clinical studies to confirm correctness of mapping the finest temperature anomalies located simultaneously in detecting areas of two or more sensors. An analogous concept, compounded however with use of different measuring and data processing techniques and exploiting a modified shape of the contact thermographic detector based on thermo-electronic sensors coupled with a specialized signal processor, was proposed by D. E. Young, C. A. Young and K. Jenkins in their patent application of 2002 (U.S. Pat. No. 6,419,636); use of an original processing algorithm for electric signals from sensors in this invention should also be supported by clinical data, for the reasons disclosed hereinabove. Another example of attempts to find a solution to the problem of adjusting a shape of a contact thermography detector to the anatomical shape of a breast, was an application for an invention filed by F. B. Asensio (ES2017374), which disclosed an apparatus moved manually on the surface of a mammary gland, comprising a thermosensor, a signal processor and a screen to depict examination results, however the employed concept could not enable direct mapping of isotherms on the surface of an examined organ; thus the predictive value of an examination based on direct discriminating analysis in search of asymmetric focal changes of a small diameter is significantly diminished.

It should be emphasized that both E. Flam in a patent application (U.S. Pat. No. 3,847,139) pertaining to liquid crystal thermographic tester incorporated in breast covering underwear, as well as Viazetti et al. in their patent (U.S. Pat. No. 3,830,224) mentioned hereinabove did not emphasize directly the meaning of direct real-time breast temperature measurement as an essential premise of the invention. Similarly, von Hollen in the already mentioned patent description (U.S. Pat. No. 6,086,247) pertaining to liquid crystal thermographic tester resembling a spread cone, did not suggest any direct reference to a continuous temperature measurement, but rather concentrated on various design features and problems with color scale interpretation and its transposition to a thermographic image. It seems that the lack of sufficient emphasis laid on critical importance of precise differentiation of thermographic changes between hypo- and hyperthermally expressed anomalies for medical thermographic diagnostics, compounded with a relative low contrast of color-temperature transitions, or repeating color responses for various temperatures, or low resolution stemming from the measurement technique used, decided on additional restrictions to the use of the above-mentioned inventions both in clinical practice as well as for screening research applications. Low resolution due to a complicated construction process of a test surface in particular, caused by forming of complex spatial shapes (as in the case of thermographic brassieres) or by trying to group separate packs containing liquid crystals into disk matrices, or by coating irregularly shaped materials with thermo-electronic sensors (as e.g. in inventions by Tumey et al., U.S. Pat. No. 5,941,832 and DeBan et al., U.S. Pat. No. 5,301,681), is a major limitation, which adds to other problems connected with an examination technique and interpretation of results leading to artifactual readouts.

Moreover, none of the above-indicated inventions employing liquid crystals did not effectively solve a problem of providing uniformity of a liquid crystal layer and obtaining high resolution to detect pathological changes of diameters below 1 mm, and especially preventing forming clusters of liquid crystals or aggregation of micelles of microencapsulated liquid crystals, and above all, suitably mechanically protecting an ultra-thin layer of liquid crystals against i.a. mechano-optical distortion.

The examination methods employed up to now, regardless of using instruments for remote or contact thermography, were based on comparative analysis, wherein readouts acquired from superficial or multipoint temperature measurements were compared with a color-temperature scale, which methods were burdened with a relatively large error, connected in the case of contact liquid crystal thermography first of all with the very readout process of examination results by comparing many component colors on a tester screen with the color-temperature scale, and in the case of contact thermography with use of thermosensor matrices, the need for each-time calibration and possible significant deviation of a locally measured temperature level from a mean for a given area and artifactually low resolution. In case of remote thermography a readout distortion is mainly due to use of temperature extrapolation methods resulting from low resolution of thermoelectronic transducer and a necessity to use software filters.

Elimination of the above-indicated problems with thermographic diagnostics was a principal objective of the invention. The invention was also designed to provide an apparatus which could be used for common screening examination made individually by women.

A thermomastographic apparatus for differentiation diagnostics designed to detect breast pathology in women according to the invention contains thermosensitive liquid crystals deposited on a flexible transparent base plate of organic polymer in a form of a film, constituting both a support and a screen. The apparatus comprises two thermographic matrices, each consisting of two test plates for simultaneous examination of both breasts in women, wherein the first matrix is used for detecting pathophysiological changes of hypothermal expression, and the second matrix is used for detecting pathophysiological changes of hyperthermal expression, in relation to physiological human temperature of 36.6° C. Each test plate is U-shaped and contains at least one monomolecular, continuous layer comprised of a homogenized mixture of liquid crystals exhibiting a thermooptical effect and sealed with elastomer.

Preferably, the matrix for detecting pathophysiological changes of hypothermal expression is used for detecting temperature changes from 31° C. to 36.6° C., and the matrix for detecting pathophysiological changes of hyperthermal expression is designed to detect temperature changes from 36.6° C. to 39° C.

Preferably, a shape of the test plate is a combination of a half of a circle, whose diameter equals half a dimension of an intermammary distance (R) vector, and a rectangle, one side of which comprises a diameter of said circle, and the second side is a calculated height of a rectangular triangle projected on an intermammary line, wherein one side of the triangle is an axillary-mammary vector (D), and the second is a ¼ of an interaxillary vector (A), said intermammary distance (R) vector is a line connecting nipples, said axillary-mammary vector (D) is an arithmetic mean of measurements of lines connecting the upper point of a left or a right axillary fold with a left or a right nipple, and said interaxillary vector (A) is a line connecting the left and right upper points of axillary folds.

Preferably the test plate has a size (TPS) calculated from the following formula:

$$T P S = 0 , 5 {π ( \frac{R}{4} )}^{2} + [ ( \frac{1}{2} R ) \times ( \sqrt{D} - \sqrt{\frac{1}{4} A} ) ]$$

wherein R, D and A have meanings given above.

Preferably a monomolecular continuous layer of liquid crystals for a matrix for detecting changes of hypothermal expression, is a liquid crystal film containing a homogenized mixture of cholesterol liquid crystals of the following formula:

cholesteryl nanonate 56-72%
cholesteryl oleylcarbonate 28-38%
cholesteryl propionata 0.5-5.0%
cholesteryl chloride 0.1-2.0%
cholesteryl benzoate 0.05-1.0%

Preferably, a monomolecular, continuous layer of liquid crystals for a matrix for detecting changes of hyperthermal expression, is a liquid crystal film containing a homogenized mixture of cholesterol liquid crystals of the following formula:

cholesteryl nanonate 57-73%
cholesteryl oleylcarbonate 27-37%
cholesteryl propionate 0.5-5.0%
cholesteryl chloride 0.1-2.0%
cholesteryl benzoate 0.05-1.0%

Preferably, the base plate is made of superficially deionized polyester.

Preferably, the continuous layer of the liquid crystal mixture is deposited by means of molecular adhesion.

Preferably, the base plate is covered with an adhesive layer, most preferably made of anionic aqueous dispersion of acrylonitrile copolymers.

Preferably, the base plate, optionally with the adhesive layer, is coated with a polymer protective coating, most preferably with a vinyl polymer, most preferably containing a chemical UV filter.

Preferably, the liquid crystal layer is coated by a sealing layer of a thermoconductive elastomer, and the sealing layer is preferably covered with an absorption layer, which layer is provided to ensure a proper background for a correct readout of thermooptical effect occurring in liquid crystals.

Preferably, the sealing layer is made of polyvinyl.

Preferably, the absorption layer is made of polyurethane.

Preferably, the absorption layer contains a black pigment, in particular micronized carbon.

Preferably, the thermographic matrices on the side contacting with skin are covered additionally by a flexible polymer layer chemically and biologically inert for human skin.

Preferably, the additional protective layer is made of a polyethylene foil or a polypropylene foil.

Preferably, the apparatus contains a handle with an examination window. The handle is provided with a permanent graphic indication of a matrix type to unequivocally read said indication in a mirror.

Preferably, the examination window has an area of from 138 cm² to 268 cm², most preferably 138 cm², 187 cm², 268 cm².

The invention provides also use of the thermomastographic apparatus for differentiation diagnostics to detect and differentiate between abnormal distributions of superficial temperature of mammary glands in women, which maps pathological changes in women's breasts, in particular changes such as cysts (cystis), fibrotic changes (degeneration fibro-cystica), inflammatory processes (mastitis), adenous type proliferation (adenoma), carcinous type proliferation (carcinoma).

Preferably, the thermomastographic apparatus is used for screening tests to detect presence of mammary gland pathology.

Use of the thermomastographic apparatus for differentiation diagnostics is characterized in that distribution of a mammary gland superficial temperature associated with corresponding color distribution on the screen of the thermomastographic apparatus for differentiation diagnostics is compared in a real time for both breasts simultaneously, at the maximum attainable resolution, obtained due to the direct and contact mapping of superficial temperature distribution by a monomolecular continuous layer of thermosensitive liquid crystals, which can detect cancerous changes in situ. According to the invention, symmetry of temperature distribution is compared simultaneously on both mammary glands by identifying areas of temperature positively different than in adjacent tissue.

Preferably, areas are identified which appear as distinctly delimited hypo- or hyper-thermal foci of stable temperature of the changes.

Preferably, comparisons are done separately for the upper lateral, upper medial, lower lateral, lower medial and perimammary part of a mammary gland.

Preferably, comparisons for the perimammary part of both breasts are done by simple application of the thermographic matrix to both breasts, wherein the upper edge of the apparatus is positioned in parallel to the interaxillary line (IAD).

Preferably, comparisons for the lower quadrants of both breasts are done by application of the thermographic matrix positioned with the lower edge of the apparatus perpendicularly to the plane of the chest, below lower edges of both breasts, followed by rotating the apparatus by 90° in the upward direction, to see on screens of the both test plates the lower quadrants of both breasts.

Preferably, comparisons for the upper quadrants of both breasts are done by application of the thermographic matrix positioned with the upper edge of the apparatus along the line connecting both coracoid processes of clavicles, to see on screens of the both test plates the upper quadrants of both breasts.

Preferably, comparisons for the medial quadrants of both breasts are done by application of the apparatus with the midpoints of the plates positioned against nipples and after drawing both examined breasts maximally aside and applying the apparatus to the breasts in such a way that both examined breasts touch with the medial quadrant surfaces the test plates, to see on screens of the both test plates the medial quadrants of both breasts.

Preferably comparisons are done after minimum 20 seconds from applying the apparatus to a mammary gland.

Preferably in case of conducting an examination individually by a patient, the examination result is read in a mirror.

Preferably, before comparing superficial temperature distribution, mammary glands are submitted to the action of a liquid medium of a temperature not greater than 10° C.

Preferably, assessment of a result of thermographic examination is based on a binomial criterion which allows for qualifying the thermographic examination as correct or pathological, on the basis of two parameters of a thermographic image, namely presence of thermal anomalies on an area of each breast, and symmetry of the anomalies, by observing whether changes are unilateral or rather occur on both breasts.

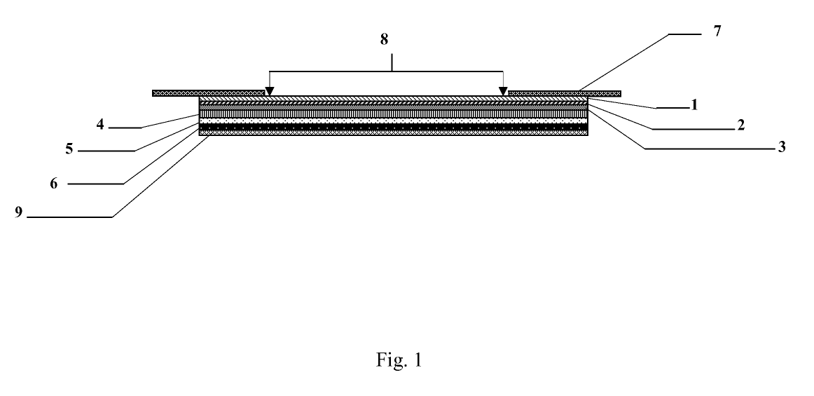

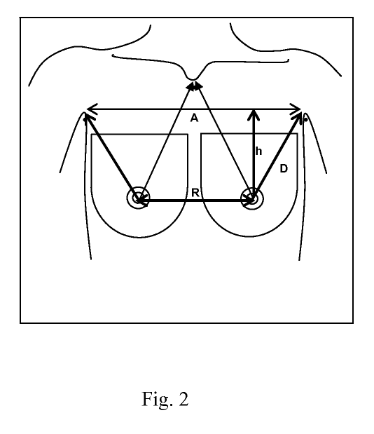

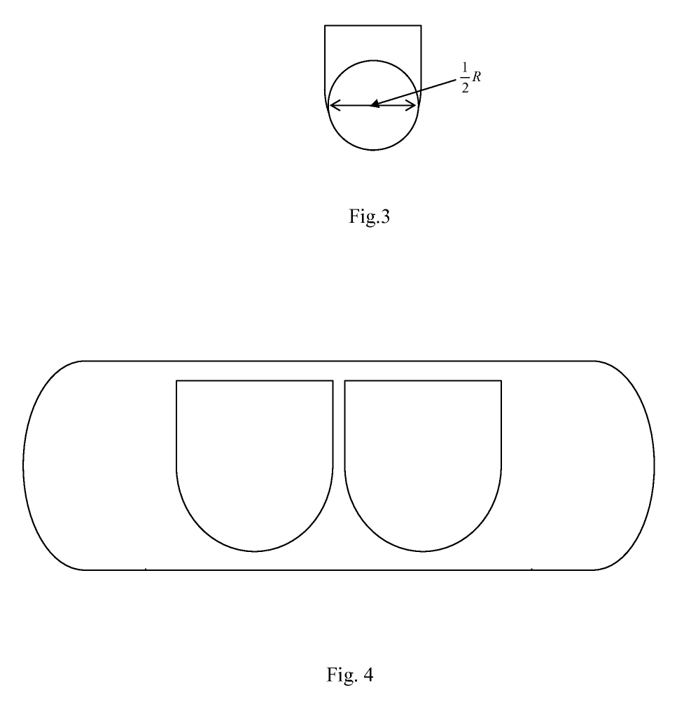

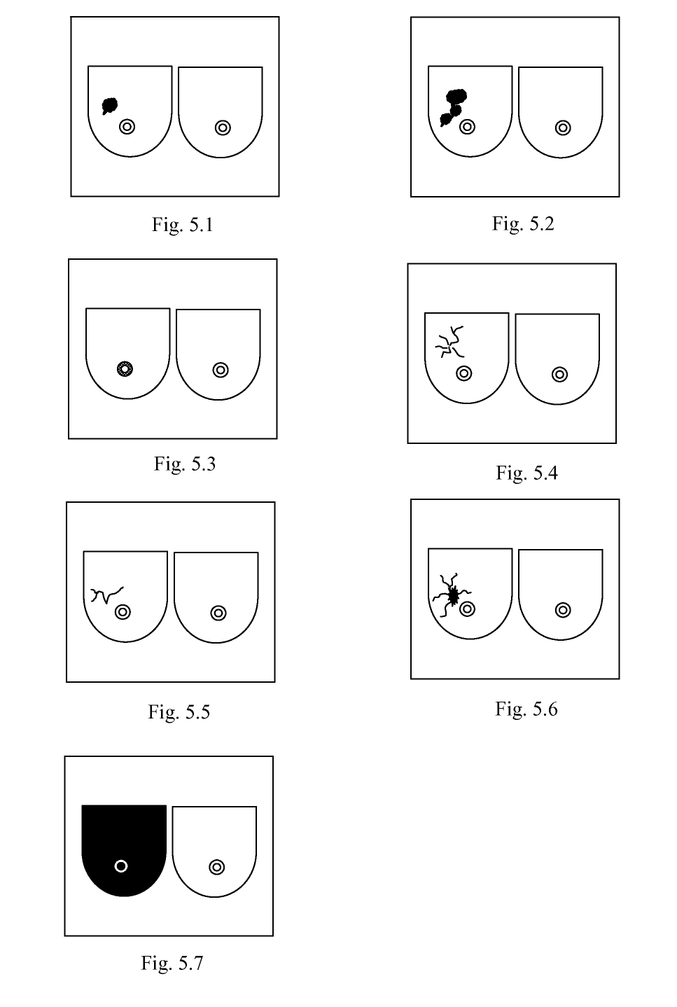

Elements of the invention as the embodiments are presented on drawings, wherein FIG. 1 shows a cross-section of a thermographic matrix, FIG. 2 shows biometric points used for determining shape and size of a test plate surface, FIG. 3 shows a combination of figures obtained from measurements forming a test plate shape, FIG. 4 shows a test plate shape, and FIG. 5 shows seven symptoms of thermal anomalies observed in a breast thermogram, which allow to classify the thermogram as pathological.

The thermomastographic matrix according to the invention represented by the embodiment of FIG. 1 of the drawings consists of the base plate 1 with the deposited adhesive layer 2, the protective coating 3, the homogenized liquid crystal layer 4, the sealing layer 5 and the absorption layer 6. From the obtained thermomastographic matrix, a tester is cut in the shape according to the invention. The base plate with successive deposited layers is fastened with a glue to the suitably profiled handle 7 with the screen 8. Preferably, the apparatus is additionally protected by the flexible polymer layer 9.

Adjusting an area of a test plate to a size of an examined mammary gland is carried out by means of an original scaling algorithm described by a mathematical formula with the use of biometric points presented on FIG. 2.

Biometric measurements of breasts are made as follows: first, a measure is established for the first and the second line of measurement, which connect a central point of a sternum incisure with left and right nipples—it is a sterno-mammary vector, a third line of measurement connects nipples—it is an intermammary vector denoted in the formula by R, a fourth line of measurement connects left and right lower points of an axillary fold—it is an interaxillary vector denoted in the formula by A, a fifth and a sixth line of measurement connects an upper point of left and right axillary folds with left and right nipples—it is an axillary-mammary vector denoted in the formula by D.

The measurements allow to calculate two major measures of a tester, lengthwise (in a mammary line) and crosswise (in a sternal line), and derived measures, followed by calculating Test Plate Correction (Enlargement) Factor denoted in the formula as PCF (Plate Correction Factor):

$$P C F = ( \frac{1}{2} R ) \times ( \sqrt{D} - \sqrt{\frac{1}{4} A} )$$

Further, optimal size of a test plate denoted by TPS (Tester Plate Size) is calculated according to the formula:

$$T P S = 0 , 5 {π ( \frac{R}{2} )}^{2} + [ ( \frac{1}{2} R ) \times ( \sqrt{D} - \sqrt{\frac{1}{4} A} ) ]$$

As a result of averaging the calculated sizes of a test plate resulted from empirical measurements and grouping them in three sections, delimited by average values of R and D, followed by employing standard deviations (SD) from means of measured breast sizes from antropometric tables, three standard sizes of test plates covering more than 98% of possible variations of breast sizes in the population of women aged above 18 were finally obtained: Size A: a plate of a test window area of 138 cm² (for a mean size R1=21 cm SD±1.2 cm), Size B: a plate of a test window area of 187 cm² (for a mean size R2=27 cm, SD±1.6 cm), Size C: a plate of a test window area of 268 cm² (for a mean size R3=30 cm SD±1.9 cm).

A single test plate, one of the two included in each thermographic matrix, comprising simultaneously a screen for reading measured results assumes, as it can be seen in FIG. 3 and FIG. 4, a U-shape, being a combination of two geometric figures, whose dimensions are derived from measurements and calculations made by a method according to the present application: a half of a circle, whose diameter equals half a dimension of an intermammary distance (R) vector, and a rectangle, one side of which comprises a diameter of said circle, and a second side is a calculated height of a rectangular triangle projected on an intermammary line, wherein one side of the triangle is an axillary-mammary vector (D), and the second is a ¼ of an interaxillary vector (A). It should be noted that ¼ is an averaged coefficient from numerous empirical measurements, with a mean deviation in the range of 10%. More precise determination of this coefficient is impossible due to excessive morphological variability of breasts and significant difficulties in obtaining reproducible results.

A thermomastographic matrix according to the invention is manufactured by homogenizing a liquid crystal mixture, preferably with a mechanical stirrer or ultrasounds. The base plate 1 is coated by the adhesive layer 2, the protective layer 3, the homogenized liquid crystal layer 4, followed by sealing with the layer 5 and coating with the absorption layer 6. From the obtained thermographic matrix, a tester is cut to the shape according to the invention. Two thermographic testers cut from the base plate for left and right breasts, are fastened with a glue from the screen side 8, further referred to as the external side, to the suitably profiled handle 7, whose size may be scaled according to a breast size, the handle being made of polymer or coated paper, and allowing to suitable place the tester plate during examination. On the screen side 8, or the external side, on the handle and about 1 cm outwards from the right tester plate, a symbol is printed corresponding to a Roman “I” or “II” to denote the adequate matrix type for examining hypo- or hyper-thermal changes. Preferably, from the elastomer layer side, after coating with all subsequent layers, the tester is additionally protected by means of thermolamination with the flexible polymer layer 9 of about 30 μm in thickness (in a form of a polyethylene or polypropylene foil) chemically and biologically inert for human skin, to allow to disinfect the test surface with polar and non-polar agents, both hydrophobic as well as based on an aqueous dispersion, used for skin disinfection.

The apparatus according to the invention allows to register temperature changes on a mammary gland surface by means of two separate matrices running under different measurement ranges from 31° C. to 36.6° C.—to register changes of hypothermal character and from 36.6° C. to 39° C.—to register changes of hyperthermal character. Imaging of superficial temperature distribution of an examined organ is carried out on a polychromatic screen with initial black background, by displaying continuous color transitions in a liquid crystal phase which correspond to individual isotherms in the given scale range. Distinction of colors is sufficient to image temperature changes of 0.2-0.5° C.

To assess thermograms in the most precise way by the differentiation method, taking into account the existence of significant individual deviations from the statistical normal distribution suitable for description of variations of superficial temperature of mammary glands in the female population, in the apparatus according to the invention a color response scale of the liquid crystal phase was coupled with a temperature scale in conditions of laboratory measurement in vitro. 6 basic thresholds in steps of 1° C. in the range from 31° C. to 36° C., and 4 basic thresholds in the range from 36° C. to 39° C. were established, and each was assigned to a specific point on a map of color distribution in a function of a temperature (a specific emission color of the visual spectrum at electromagnetic wave length from 400 nm to 760 nm), or a specific color visible on a tester screen.

Directly coating the deionized base plate (support) surface by monomolecular continuous homogenized layer of a liquid crystal mixture allows to cover homogenously the entire tester surface, but first of all does not produce artifactual granular structures influencing thermogram readout quality.

It should be emphasized that distance between breasts has a minor impact on size of the apparatus according to the invention in accordance with population and anatomical variability, due to the use, in the universal scaling algorithm, the intermammary distance vector, which is subsequently divided into halves, with the a priori assumption of a zero distance between breasts—to make an examination easier, test plates for left and right breasts are flexibly connected with an interval of 5 mm, which is compensated by the examination technique.

Use in the apparatus according to the invention a two-interval continuous analysis method for real time imaging of isotherm distribution on an examined surface of a mammary gland, on a screen of a readout area of at least 138 cm², at the theoretic resolution of the order of one molecule and very high contrast of color-temperature transition of the liquid crystal mixtures used, with gradation of 0.5° C., allows to avoid a majority of artifactual readouts connected with:

i*) low thermographic image resolution,
ii*) low contrast between color transitions for particular isotherms.

The method of thermographic examination employed in the invention, which avoids use of additional power sources and a graphical processor, allows to employ the apparatus for carrying examination in ambulatory and home circumstances, the same being practically impossible in case of devices based on IR-sensitive cameras.

Uniqueness of the diagnostic method lies also in the fact that interpretation of a thermographic result is not based on referring revealed images to a color-temperature scale, but on a simple discriminating (differential) observation to find an appearance or the lack of thermal anomalies in a form of asymmetric (visible on one breast only) and distinctly isolated areas of different color, corresponding to a temperature different than in surrounding tissue, said examination being not designed to quantify temperature differences at the changes, but only identifying them as hypo- or hyper-thermal.

Utility of the apparatus according to the invention has strictly a screening character, meaning that the examination is designed to identify early thermographic risk factors of breast cancer occurrence; therefore it is basically not designed for establishing an anatomical localization of pathological changes, but rather for functional imaging of occurrences of thermal anomalies which can be predictive for various pathologies of mammary glands.

In use for screening breast pathology medical diagnostics, the apparatus comprising the liquid crystal thermographic tester, due to a coupled color-temperature scale, is perfectly fit for both fast and objective assessment of thermal anomalies without the necessity for quantification of a local temperature of the anomaly; clearly visible differences for particular color transitions which correspond to temperature variations with gradation below 1° C., in an entire measurement scale both for hypo- and hyper-thermal analysis matrix.

Examination may be done individually by a patient at the mirror. Each time a patient examines only three parameters of a thermographic image:
1. presence of anomalies in an area of both examined breasts (presence of distinctly distinguishable foci of a color/temperature different than in surrounding tissue),
2. symmetry of thermographic images for a left and right breast (the observed changes being unilateral or occurring in both breasts),
3. a thermal diversity degree of a thermogram (presence of many colors or prevalence of only one color)

Due to the 3-stage evaluation of a thermogram the examination is simple, and allows simultaneously to establish precisely the presence of presumable thermographic anomalies, being the only indicator (or a screening marker) which indicates necessity of a visit to a specialist to verify the examination result and confirm or exclude suspicions of existing pathology of a mammary gland. Since the presently claimed examination methodology assumes repeating every test which revealed any thermographic anomalies after minimum 30 minutes and after 2 days, it can be assumed that such redundancy constitutes a sufficient filter to prevent revealing an excessive number of false positive results stemming from a high sensitivity of the employed technique.

In spite of existence of many complex algorithms for analysis of thermographic images, specificity of inventive liquid crystal mixtures allows for an autonomous and fast (about 20 seconds) and unequivocal readout of a temperature distribution, along with clear distinction of foci of a temperature different than in surrounding tissue (separately for hypo- and hyper-thermal changes). This is the key and sole differentiating criterion in thermographic screening examination.

The invention, which restricts a diagnostic procedure of contact thermography to a differentiation test, allows to use the apparatus at home, being a qualitative change in a present approach to a problem of early breast cancer diagnostics. Women did not have so far any objective method of scientifically confirmed efficacy for conducting completely non-invasive, harmless, cheap, voluntarily repeatable breast examination, being additionally a method comprising unusually early breast pathology marker, significantly increasing chances for full recovery.

If thermographic examination made with two matrices marked by “I” and “II”, and serving to detect hypo- and hyper-thermal changes respectively, reveal presence of any thermographic anomaly, even single, of a clearly visible focus or foci, or clusters of a color different than surrounding tissue, which image specific temperatures and correspond accordingly to hypo- or hyper-thermal pathology, the examination should be repeated after about 30 minutes and after 2 days. If the originally observed thermographic changes are revealed also in the two subsequent examinations, it is the absolute indication for medical consultation and further optional specialistic differential diagnostics.

Conversely, a negative result of thermographic examination, due to a high sensitivity of the method indicated in the references, does not require any confirmation by other specialized diagnostic procedure, with a proviso that the above does not concern women belonging to genetic or age risk groups, where advisability of use of associated examination techniques employing various imaging techniques and/or immunological or genetic tests should be considered.

Examination with the apparatus according to the invention is conducted in a room, in optimal illumination and temperature conditions. The conditions are considered met in the daylight or artificial illumination and minimal light intensity of 300 1× and mean air temperature kept in the range of 20° C. to 24° C. Examination may be conducted in a standing or sitting position. Skin on an examined surface of a breast should be dry. An apparatus for thermomastographic examination should be selected according to breast size and should be applied in a way to embrace by examination all quadrants of both mammary glands.

When examination is done individually by a patient, and examination results are read in a mirror, the mirror should stand at a distance of at least 30 cm, and no more than 90 cm for a screen a thermographic image appears on.

Examination starts with applying to both breasts at once the thermographic matrix for detecting hypothermal changes, marked with a Roman “I” on the handle. After about 20 seconds a thermographic image is stabilized and a result of the examination can be read, or local thermographic anomalies looked for. Examination with the matrix for detecting hypothermal changes, marked with a Roman “I” on a handle, is repeated four times for each position of a tester plate provided in the instruction. A result is written on a thermographic examination card: a negative one in case of the lack of thermographic anomalies, and a positive one in case of their presence.

A thermogram contained within a norm indicates a negative result of thermographic examination (for every matrix denoted “I” and “II”)—a thermographic norm means the lack of thermal anomalies, both of hypo- and hyperthermal expression: no focal asymmetric thermal anomalies were found. Examination does not require actually any other clinical-diagnostic verification by other imaging techniques and/or invasive diagnostics.

A pathological thermogram means a positive result of thermographic examination (for a matrix denoted “I” or “II”)—thermal anomalies were found in thermograms of both hypo- and hyperthermal expression, in a form of presence of asymmetric thermal changes, symmetric, occurring in only one breast. Examination needs urgent clinical verification within a month, also by other imaging method and/or invasive diagnostics.

To consider examination contained in a thermographic norm, a negative result from both matrices is necessary; to consider examination pathological, a positive result for the first or second matrix is enough.

Further, regardless of detecting hypothermal changes with the matrix, marked with a Roman “I” on the handle, on any of the four thermographic images, a second examination with the matrix for detecting hyperthermal changes, marked with a Roman “II” on the handle. Examination with the matrix for detecting hyperthermal changes is done analogously, an image is read after stabilizing for about 20 seconds and repeated in 4 exposures, and a result is noted: a negative one in case of the lack of thermographic anomalies, and a positive one in case of their presence.

To facilitate observations of thermal anomalies on thermomastograms, a chart of patterns of thermal anomalies was elaborated comprising seven basic symptoms of thermal anomalies noticeable on a breast thermogram, which permits one to classify a thermogram as pathological. The chart is only supportive and is illustrated on FIG. 5 of the drawings, wherein the pattern 5.1 shows a single focal change, the pattern 5.2 shows a multifocal change, the pattern 5.3 shows perimammary and perimammillar changes, the pattern 5.4 shows a vasogenic concentrated change, the pattern 5.5 shows a vasogenic linear change, the pattern 5.6 shows a vasogenic composite change, the pattern 5.7 shows a general asymmetrical thermal change.

An occurrence of any of the seven symptoms of thermal anomalies illustrated on the chart on a thermomastographic image causes recognition of the thermogram as pathological, with occurring of any change of a temperature significantly higher or lower temperature than in surrounding tissue in the case of isolated unsymmetrical change, appearing only in an area of one breast, being a sufficient premise to consider the result of examination not meeting the requirements for a thermographic standard.

If in thermographic examination repeated after 30 minutes no previously observed thermal anomalies appear, an additional examination is recommended exploiting a technique of so called “cold stress” (“cold-stress test”), comprising subjecting both examined breasts to a brief (about 5 seconds) influence of cold water cooled to about 10° C. The “cold stress” is designed to suppress a vasodilatory effect originating in regular vascularization, which is sensitive to structural factors, as opposed to pathological vascularization, which is less sensitive or insensitive to this kind of a stressor.

A technique for thermomastographic examination with use of the apparatus according to the invention is described below.

1. The first technique—simple examination comprising:
a.) positioning the apparatus—the inner surface, without a surprint, must be directed to examined breasts, the outer surface, marked by “I” or “II”, and with a visible screen should be directed to a face of an examining physician, or to a mirror, if the examination is done by a patient,
b.) grabbing the apparatus comprising two diagnostic plates for examining a left and a right breast, by both handles and straightening it to the entire width,
c.) lifting the apparatus to a breast level, to position the center of each plate against a nipple,
d.) applying the apparatus to breasts without shifting it sidewise or up, or down. The thrust of the apparatus should be selected to permit a good contact of both test plates with surfaces of examined breasts, without exerting pressure or producing discomfort to the patient,
e.) holding the apparatus in the examination position for about 20 seconds to stabilize the obtained thermographic image,
f) reading the result of thermographic examination, in succession:
   the left breast: the upper half, the lower half
   the right breast: the upper half, the lower half
   comparing thermograms for the left and right breasts to find asymmetries of thermographic anomalies,
      2. The second technique for lower quadrant examination comprising:
a.) positioning the apparatus—the inner surface, without a surprint, must be directed to examined breasts, the outer surface, marked by “I” or “II”, and with a visible screen should be directed to a face of an examining physician, or to a mirror, if the examination is done by a patient,
b.) grabbing the apparatus comprising two diagnostic plates for examining a left and a right breast, by both handles and straightening it to the entire width,
c.) lifting the apparatus positioned in parallel to the chest, to a level of lower ribs, for a lower edge of the apparatus to be positioned on the level of lower breast squares,
d.) applying the apparatus to breasts in such a way that the apparatus is positioned perpendicularly to a chest surface, and both examined breasts are arranged loosely by lower quadrants of the tester plates, than slowly raise the apparatus upwards and rotate in a direction of the chest, pushing it simultaneously to the both breasts and lifting them also gently upwards, for the tester screens to be visible for the examining physician, or appear in a mirror, if the examination is done by a patient,
e.) holding the apparatus in the examination position for about 20 seconds to stabilize the obtained thermographic image,
f.) reading the result of thermographic examination, in succession:
g.) the left breast: the lower half
h.) the right breast: the lower half
i.) comparing thermograms for the left and right breasts to find asymmetries of thermographic anomalies
   3. The third technique for upper quadrant examination comprising:
a.) positioning the apparatus—the inner surface, without a surprint, must be directed to examined breasts, the outer surface, marked by “I” or “II”, and with a visible screen should be directed to a face of an examining physician, or to a mirror, if the examination is done by a patient,
b.) grabbing the apparatus comprising two diagnostic plates for examining a left and a right breast, by both handles and straightening it to the entire width,
c.) lifting the apparatus positioned in parallel to the chest, to a level of clavicles, for an upper edge of the apparatus to be positioned on the level of clavicles,
d.) applying the apparatus to breasts without shifting it sidewise or up, or down, for upper quadrants of both examined breasts were covered by the tester plates. A good contact of both test plates with surfaces of examined breasts should be secured, without exerting pressure or producing discomfort to the patient,
e.) holding the apparatus in the examination position for about 20 seconds to stabilize the obtained thermographic image,
f.) reading the result of thermographic examination, in succession:
g.) the left breast: the upper half
h.) the right breast: the upper half
i.) comparing thermograms for the left and right breasts to find asymmetries of thermographic anomalies
   4. The fourth technique for medial quadrant examination comprising:
a.) positioning the apparatus—the inner surface, without a surprint, must be directed to examined breasts, the outer surface, marked by “I” or “II”, and with a visible screen should be directed to a face of an examining physician, or to a mirror, if the examination is done by a patient,
b.) grabbing the apparatus comprising two diagnostic plates for examining a left and a right breast, by both handles and straightening it to the entire width,
c.) lifting the apparatus to a breast level, to position the center of each plate against a nipple,
d.) shifting both examined breasts maximally apart and applying the apparatus to breasts for both examined breasts to touch the tester plates with the entire surfaces of medial quadrants, than slowly pushing the apparatus towards both breasts for the tester screens to be visible for the examining physician, or appear in a mirror, if the examination is done by a patient,
e.) holding the apparatus in the examination position for about 20 seconds to stabilize the obtained thermographic image,
f.) reading the result of thermographic examination, in succession:
g.) the left breast: the medial half
h.) the right breast: the medial half
i.) comparing thermograms for the left and right breasts to find asymmetries of thermographic anomalies

An algorithm for thermomastographic examination with the apparatus according to the invention assumes that the four subsequent techniques for positioning the apparatus should be applied for examination of breasts with two thermographic matrices marked “I” and “II” for detecting changes of hypo- and hyperthermal, respectively.

The invention is presented in detail in the following working examples.

### EXAMPLE 1

The apparatus according to the invention comprises two thermographic matrices, each comprising two test plates of a cross-section as shown in the example on FIG. 1 of the drawings. The test plate comprises a base plate 1 coated with an adhesive layer 2, a protective layer 3, a layer of homogenized liquid crystals 4, a sealing layer 5 and an absorption layer 6. From the thermographic matrix such obtained a tester shaped according to the invention is cut. The base plate covered with subsequent layers is fastened with a glue into a suitably profiled handle 7 with a screen 8. Preferably, the apparatus is additionally protected by a flexible polymer layer 9.

The base plate 1 is made of transparent organic polymer in the form of a polyester foil of a thickness of 80 μm.

The apparatus is manufactured first by deionization of a polyester foil sheet to be the base plate 1, by means of an electric deionizer (eg. the ION Virtual AC Inteligent Static Neutralizer apparatus). Then, the foil sheet is placed on a metallic grounded table provided with a vacuum stabilizer, which is subsequently covered by layers necessary to manufacture the proper thermomastographic tester.

The deionized polyester foil sheet is coated with the adhesive layer 2, comprising an anionic aqueous dispersion of copolymers comprised of acrylic acid cyanoester (acrylonitrile) deposited by a roller method, then an adhesive layer 2 is dried at the temperature up to 80° C. by means of IR radiators for about 20 minutes.

The dried adhesive layer 2 is covered by about 50 μm of the protective layer 3 of a vinyl polymer comprising a chemical UV filter containing: polyvinyl alcohol, acetone, 40% aqueous solution of formaldehyde, ethanol, glycerol, sulphonic acid phenylbenzimidazolate, nonoxinol, demineralized water. The mixture containing the vinyl polymer is prepared in a chemical reactor under the atmospheric pressure and mechanically homogenized by means of laminar stirrers rotating at a frequency of from 500 to 1000 Hz. The deposited protective layer 3 is dried at a temperature below 80° C. by means of IR radiators for about 20-30 minutes.

After reaching a tack-free state by the protective layer 3 the continuous liquid crystal layer 4 of a homogenized mixture of liquid crystals is deposited. Coating is made by means of molecular adhesion of an ultrathin continuous layer of the liquid crystal film 4 to the surface of the polyester foil constituting the base plate 1. During coating, it is necessary to technologically link processes of homogenization and coating, because the prepared liquid crystal emulsion must be consumed within 48 hours. Homogenization of a mixture of particular liquid fractions proceeds at the temperature from 21° C. to 24° C., and initial homogenization is done by means of mechanical sieve stirrers rotating optimally at a frequency of from 40 to 1000 Hz for about 2 hours. The proper homogenization is done by means of an ultrasonic homogenizer (e.g. MP250 of Hielscher). The homogenized mixture of liquid crystals is controlled viscometrically (e.g. in a capillary viscometer LK.2.2 of Rheotest). Deposition of the homogenized liquid crystal mixture of the proper density on a dried protective layer is performed by means of a calibrated gravitational dispenser positioned by a pneumatic-hydraulic system, to avoid direct touching the surface of the polyester foil. To provide uniform thickness and continuity of the deposited liquid crystal film, a maximum distance between a lower edge of the dispenser and the surface of the polyester foil is from 0.05 to 0.15 mm and is controlled by an optoelectronic sensor based on a solid state laser. The deposited continuous liquid crystal layer 4 is subsequently dried by means of IR radiators at a temperature below 80° C. for about 30 minutes.

Composition of a mixture of cholesteric liquid crystals is determined according to the following weight proportions:

For a matrix marked by “I” for detecting changes of hypothermal expression:

57.2%—cholesteryl nanonate (pelargonate);
28.4%—cholesteryl oleylcarbonate;
1.7%—cholesteryl propionate;
1.2%—cholesteryl chloride;
0.5%—cholesteryl benzoate;
1.0%—4,4′ dipentylazoxybenzene.

For a matrix marked by “II” for detecting changes of hyperthermal expression:

71%—cholesteryl nanonate (pelargonate);
32.5%—cholesteryl oleylcarbonate;
0.5%—cholesteryl propionate;
0.12%—cholesteryl chloride;
0.07%—cholesteryl benzoate;
2.0%—4,4′ dipentyloazoksybenzen.

Onto the dried liquid crystal layer 4 the sealing layer 5 is deposited with the following chemical composition: polyvinyl alcohol up to 20%, acetone, 40% aqueous solution of formaldehyde, ethanol, glycerol, nonoxinol, demineralized water. Deposition is performed by means of a grawitational dispenser, without a mechanical contact with the unprotected liquid crystal layer extremely sensitive to mechanical damages. Due to tixotropic properties of the mixture containing liquid elastomer, uniform and thorough distribution thereof onto the entire covered surface of liquid crystals proceeds without employing additional procedures. After coating with the semiliquid sealing layer 5, during about 60 minutes of its polymerization and drying at a temperature below 80° C. by means of IR radiators, the entire surface of the liquid crystal film 4 is finally covered with a polymer forming directly in statu nascendi, leaving no unprotected surface.

Onto the dried sealing layer 5 the absorption layer 6 is deposited, which is composed of the mixture of aliphatic and aromatic polyurethanes up to 50% and modifiers comprising ethanol 2-1-methylethoxyacetate and 2-propoksyethanol, with addition of a pigment comprising micronized chemically pure carbon, compatible with No 77.266 according to Colour Index International, in a dispersion containing organic solvents comprising aliphatic and aromatic hydrocarbons, which layer is designed to provide proper background for correct reading of thermooptic effect occurring in liquid crystals.

After coating and reaching a tack-free state of the absorption layer 6 mechanical or laser punching occurs, wherein from a rectangular polyester foil sheet coated with particular layers, a proper shape of two base plates of the thermographic tester for examining a left and a right breast is punched, in one of three basic sizes with the following surfaces: size A—a plate of the test window surface up to 138 cm², size B—a plate of the test window surface of 187 cm², size C: a plate of the test window surface of 268 cm².

The two punched test plates comprise an essential working element of the apparatus. To secure a full functionality of the apparatus, it is necessary to fasten them, by means of a cyanoacrylic glue, or other glue used to glue polyester, in a properly prepared handle 7 made of elastic inextensible polymer or coated paper, whose size is scaled depending on a plate size; wherein at the side of a visible window of the screen 8, a symbol “I” is printed to mark a matrix for detecting changes of hypothermal expression or “II” to mark a matrix for detecting changes of hyperthermal expression.

The last step in the manufacture of the thermomastographic apparatus following gluing of both base plates in the handle 7 is covering the apparatus, and namely the handle and both base plates at the side contacting with the patient's skin, with the polymer contact layer 9 with a thickness up to 30 μm, in the form of a polyethylene or polypropylene foil deposited by means of a thermolamination process at a temperature below 80° C., chemically and biologically inert for human skin, and providing for disinfection of a test surface by means of polar and non-polar agents, both hydrophobic and based on an aqueous dispersion used for skin disinfection.

### EXAMPLE 2

### Scaling of the Apparatus

In the apparatus of Example 1, a color scale of a response of a liquid crystal phase was coupled with a temperature scale in conditions of a laboratory measurement in vitro. The six basic thresholds, at intervals of 1° C., in the range of 31° C. to 36° C., for a matrix for detecting changes of hypothermal expression, and four basic thresholds in the range of 36° C. to 39° C., for a matrix for detecting changes of hyperthermal character, were established empirically, each said threshold assigned to a particular point on a map of color distribution against the temperature, or a particular emission color of the visual spectrum at the electromagnetic wave lengths of 400 nm to 760 nm, comprising a particular color visible on a screen of a tester, as shown in Table 1 and 2.

TABLE 1
Color scale for a matrix for detecting hypothermal changes with 6
reference points corresponding successively to temperatures [T ° C.]
determined with a step of 1° C. in the range of 31° C. to 36° C.:

| | Electromagnetic | Color scale according to the |
|---|---|---|
| Temperature | wave length (λ) | invention, preferably with 6 |
| [° C.] | [nm] | reference points |
| 31° C. | 755.84 nm | brown-red color |
| 32° C. | 670.9 nm | orange-red color |
| 33° C. | 609.35 nm | yellow-orange color |
| 34° C. | 563.4 nm | green-yellow color |
| 35° C. | 527.53 nm | green color |
| 36° C. | 499.48 nm | blue color |

TABLE 2
Color scale for a matrix for detecting hyperthermal changes with 4
reference points corresponding successively to temperatures [T ° C.]
determined with a step of 1° C. in the range of 36° C. to 39° C.

| | Electromagnetic | Color scale according to the |
|---|---|---|
| Temperature | wave length (λ) | invention, preferably with 4 |
| [° C.] | [nm] | reference points |
| 36° C. | 645.2 nm | orange color |
| 37° C. | 515.06 nm | green color |
| 38° C. | 451.95 nm | blue color |
| 39° C. | 420.78 nm | violet color |

The color scales allow a person reading the examination result, what is a dominant range of the visual spectrum, or a color of reflected light, observed during the examination on the tester's screen, corresponding to a particular temperature measured by the tester on a surface of breasts, as well as a direction of thermal anomaly shift towards higher or lower temperature compared to surrounding tissue. Though the above scales are discrete in character, due to optical effects occurring in the liquid crystal phase in the apparatus according to the invention a continuous analysis of real time temperature distribution in an examined organ, in a full temperature scale, the sufficient contrast of color transitions permitting to register thermal changes of even 0.2° C. In applications for screening medical diagnostics of breast pathology, the liquid crystal thermographic tester apparatus, due to the coupled color-temperature scale is suitable to fast and at the same time objective assessment of presence of thermal anomalies, without the need for determining a numeric value of a local temperature of such anomaly because of clearly visible differences for particular color transitions which correspond to temperature variations with gradation below 1° C., with gradation below 1° C., in an entire measurement scale a matrix for analysis of pathological changes of hypo- and hyper-thermal expression.

What is claimed is:

1. A thermomastographic apparatus for differentiating diagnostics for detecting breast pathology in women composed of a first set and a second set of thermographic screens, each of the first and second set comprising:
a flexible, transparent, base-plate of organic polymer in a form of foil, being at the same time a support and a screen, and
two test-plates for simultaneous examination of both breasts of a woman,
said first set is designed to detect pathophysiological changes of hypothermal expression, and
said second set is designed to detect pathophysiological changes of hyperthermal expression, relative to a physiological temperature of a human body 36.6° C. and
each test-plate being U-shaped and containing at least one continuous layer being formed from a homogenized mixture of thermotropic liquid crystals,
whereat each base-plate is covered sequentially with an adhesive layer, next with a protective layer, next at least with the one, continuous layer being formed from thermotropic liquid crystals, next with a sealing layer and an absorption layer containing black absorptive pigment, whereat the continuous layer containing thermotropic liquid crystals exhibiting features of a thermo-optically active mesophase is applied in a form of nongranular coating directly on the surface of the protective layer on basis of the molecular adhesion effect.

2. The thermomastographic apparatus of claim 1, wherein the first set of thermographic screens is dedicated to display of pathological changes in mammary glands of hypothermal expression at the temperature of from 31° C. to 36.6° C.

3. The thermomastographic apparatus of claim 1, wherein the second set of thermographic screens is dedicated to display pathological changes in mammary glands of hyperthermal expression at the temperature of from 36.6° C. to 39° C.

4. The thermomastographic apparatus of claim 1, wherein the geometric shape of the test-plate that constitute the thermographic screen, is defined by an algorithm arising from a geometrical superposition of a half of a circle, the circle's diameter equals ½ a dimension of an intermammary distance (R) vector, and a rectangle, one side of the rectangle comprises a diameter of said circle, and a second side of the rectangle is a calculated height of a rectangular triangle projected on an intermammary line, wherein one side of the triangle is an axillary-mammary vector (D), and the second side of the triangle is a ¼ of an interaxillary vector (A), said intermammary distance (R) vector is a line connecting nipples, said axillary-mammary vector (D) is an arithmetic mean of measurements of lines connecting the upper point of a left or a right axillary fold with a left or a right nipple, and said interaxillary vector (A) is a line connecting the left and right upper points of axillary folds.

5. The thermomastographic differentiation apparatus of claim 4, wherein a test-plate size (TPS) is calculated from the formula:
$$T P S = 0 , 5 {π ( \frac{R}{4} )}^{2} + [ ( \frac{1}{2} R ) \times ( \sqrt{D} - \sqrt{\frac{1}{4} A} ) ]$$

6. The thermomastographic apparatus of claim 1 wherein the base-plate is made of superficially deionized polyester.

7. The thermomastographic apparatus of claim 1, wherein the adhesive layer is made of an anionic aqueous dispersion of acrylonitrile copolymers.

8. The thermomastographic apparatus of claim 1, wherein the protective layer is formed of a vinyl polymer.

9. The thermomastographic apparatus of claim 1, wherein the protective layer contains a chemical UV filter.

10. The thermomastographic apparatus of claim 1, wherein the sealing layer is formed from a thermoconductive elastomer.

11. The thermomastographic apparatus of claim 1, wherein the absorption layer is formed from a polyurethane polymer.

12. The thermomastographic apparatus of claim 1, wherein the thermographic screens on the side contacting with a patient's skin are covered additionally by a chemically and biologically neutral flexible polymer protective layer.

13. The thermomastographic apparatus of claim 12, wherein the additional polymer protective layer is made of a polyethylene foil or a polypropylene foil.

14. The thermomastographic apparatus of claim 1, further comprising a handle with an examination window.

15. The thermomastographic apparatus of claim 14, wherein the handle contains a graphic indication of a thermographic screen type allowing the screen type's identification also in the mirror view.

16. A method for detecting and differentiating between abnormal distributions of superficial temperature of mammary glands in women imaging pathological changes, in particular for detection and differentiation of pathologies on such as hypothermic and hiperthermic nature, particularly use of the screen for imaging of the hypothermic pathologies for detection and differentiation of the changes of non-neoplastic character like cysts (cystis), fibrotic changes (degeneration fibro-cystica), and use of the screen for imaging of the hiperthermic pathologies for detection and differentiation of the changes of potentially neoplastic character like: glandular type proliferation (adenoma), carcinous type proliferation (carcinoma) and inflammatory processes (mastitis), utilizing the thermomastographic apparatus of claim 1.

17. The method of claim 16, further for screening tests to detect presence of mammary gland pathology which presence is evidenced in bivalent scale.

18. The method of claim 16, wherein distribution of a mammary gland superficial temperature associated with corresponding color visible on the particular type of a thermographic screen for the purpose of differentiation diagnostics, is compared in a real time, for both breasts simultaneously, at the maximum attainable optical resolution, obtained due to the direct in a scale 1:1 and contact mapping of superficial temperature distribution by a molecular continuous layer being formed from thermotropic liquid crystals exhibiting features of a thermo-optically active mesophase, which allows to detect changes generated by in situ cancer.

19. The method of claim 16, wherein for the purpose of taking examination's reading, symmetry of temperature distribution is compared simultaneously on both mammary glands by identifying areas of temperature significantly different than in adjacent tissue.

20. The method of claim 19, wherein thermo-pathological areas are identified and differentiated as areas which appear as noticeably distinguished foci of hypo- or hyper-thermal nature in relation to a physiological temperature of a human body 36.6° C. and which become visible on a thermographic image as anomalies with stable temperature.

21. The method of claim 16, wherein comparisons are done separately for the upper lateral, upper medial, lower lateral, lower medial and perimammary part of a mammary gland.

22. The method of claim 21, wherein comparisons for the perimammary part of both breasts are done by simple application of the thermographic screen to both breasts, wherein the upper edge of the apparatus is positioned in parallel to the interaxillary line (IAD).

23. The method of claim 21, wherein comparisons for the lower quadrants of both breasts are done by application of the thermographic screen positioned with the lower edge of the apparatus perpendicularly to the plane of the chest, below lower edges of both breasts, followed by rotating the apparatus by 90° in the upward direction, to see on screens of the both test plates the lower quadrants of both breasts.

24. The method of claim 21, wherein comparisons for the upper quadrants of both breasts are done by application of the thermographic screen positioned with the upper edge of the apparatus along the line connecting both coracoid processes of clavicles, to see on screens of the both test plates the upper quadrants of both breasts.

25. The method of claim 21, wherein comparisons for the medial quadrants of both breasts are done by application of the apparatus with the midpoints of the plates positioned against nipples and after drawing both examined breasts maximally aside and applying the apparatus to the breasts in such a way that both examined breasts touch with medial quadrant surfaces of the test plates, to see on screens of the both test plates the medial quadrants of both breasts.

26. The method of claim 16, wherein comparisons are done after about a minimum of 20 seconds from applying the apparatus to a mammary gland.

27. The method of claim 16, wherein examination is conducted personally by patient and the examination result is read in a mirror.

28. The method of claim 16, wherein before repeated comparison of superficial temperature distribution, mammary glands are submitted to stimulation by vasoconstrictive stressor being a liquid medium of a temperature not greater than 10° C.

29. The method of claim 16, wherein an assessment of a result of thermographic examination is based on a binomial criterion which allows for qualifying the thermographic examination as correct or pathological, on the basis of two parameters of a thermographic image comprising a presence/or not of thermal anomalies on an area of each breast, and symmetry of these anomalies, by observing whether changes are unilateral or rather occur on both breasts.
